(19) United States
(12) Patent Application Publication
(10) Pub. No.: US 2006/0229597 A1
(43) Pub. Date: Oct. 12, 2006

(54) Ultrasound medical device and related methods of use

(76) Inventors: Jon T. Mcintyre, Newton, MA (US); Isaac Ostrovsky, Wellesley, MA (US); Todd Sloan, Medway, MA (US); Jeffrey P. Cheek, Hopkinton, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/100,517

(22) Filed: Apr. 7, 2005

(51) Int. Cl. A61B 18/18

(52) U.S. Cl. 606/41

(57) ABSTRACT

An embodiment of the invention includes providing a medical device including an elongate portion and a distal portion, the distal portion including a plurality of substantially flat crystals configured to emit energy to a common focal point. The medical device may be advanced into a body lumen, energy may be provided to a body tissue via the substantially flat crystals, and the medical device may be removed from the body lumen.

### DESCRIPTION OF THE INVENTION

1. Field of the Invention

An embodiment of the invention is related to a medical device for delivering energy to body tissue. The energy may be delivered using ultrasound emitted from flat crystals. The medical device may also be configured to accommodate an additional medical device, for example, a fiber optic device.

2. Background of the Invention

Ultrasound devices may be used as a diagnostic or therapeutic device in medical procedures. For example, ultrasound devices may be used for medical imaging of internal organs and/or may be used to treat body tissue, such as the removal of undesirable tissue. Ultrasound devices may be expensive, limiting the ability of such devices to be disposable. Reusable ultrasound devices, however, suffer from sterility problems. Contamination of a reusable ultrasound device can cause health risks to the patient.

### SUMMARY OF THE INVENTION

An embodiment of the invention includes a medical device including an elongate portion and a distal portion including a plurality of substantially flat crystals configured to emit energy to a common focal point.

In various embodiments, the invention may include one or more of the following aspects: the plurality of substantially flat crystals may be arranged on a concave surface of the distal portion; the distal portion may include a sonolucent cover; the sonolucent cover may cover the plurality of substantially flat crystals; the common focal point may be located adjacent to a side of the distal portion; the common focal point may be located distal to the distal portion; the distal portion may define a cavity for containing a fluid; the elongate portion may include a lumen configured to provide fluid to the cavity; the sonolucent cover may define at least a portion of the cavity; a concave surface of the distal portion may define at least a portion of the cavity, the plurality of substantially flat crystals being arranged on the concave surface; the elongate portion may include a lumen configured to accommodate a fiber optic device; a distal end of the lumen may be disposed on a side of the elongate portion; a fiber optic device disposed in the lumen; an area around the distal portion may be visible via the fiber optic device; the energy may be ultrasound energy; the elongate portion may be flexible so as to traverse tortuous anatomy of a patient; the plurality of substantially flat crystals may be arranged in a two-dimensional array; and the plurality of substantially flat crystals may be arranged in a three-dimensional array.

Another embodiment of the invention may include a medical device including a distal portion including a plurality of discrete substantially flat crystals configured to emit ultrasound energy to a common focal point to treat the tissue, a proximal portion configured to control the emission of ultrasound energy, and an elongate flexible portion connecting the proximal portion to the distal portion.

A further embodiment of the invention may include a method of treating tissue including providing a medical device including an elongate portion and a distal portion, the distal portion including a plurality of substantially flat crystals configured to emit energy to a common focal point, advancing the medical device into a body lumen, providing energy to a body tissue via the substantially flat crystals, and removing the medical device from the body lumen.

In various embodiments, the invention may include one or more of the following aspects: the medical device may include a fiber optic device, and may further comprise visualizing an area around the distal portion via the fiber optic device; providing a fiber optic device; advancing the fiber optic device through a lumen in the elongate portion; visualizing an area around the distal portion via the fiber optic device; determining an area of body tissue to be treated; positioning the medical device in the body lumen so as to deliver energy to the area of body tissue to be treated; the positioning may occur during the visualizing; the energy may be provided to an area on a surface of the body tissue; the energy may be provided to an area below a surface of the body tissue; the energy may be provided to an area distal to the distal portion; the energy may be provided to an area proximal to a distalmost point of the distal portion; the energy may be focused on the area of body tissue to be treated; adjusting a position of the plurality of flat crystals relative to other portions of the distal portion while the medical device is disposed in the body lumen; the energy may be ultrasound energy; the plurality of substantially flat crystals may be disposed in a cavity of the medical device; providing fluid to the cavity; the distal portion may include a sonolucent cover; the sonolucent cover may cover the plurality of substantially flat crystals; the energy emitted by the plurality of flat crystals may travel through the sonolucent cover prior to exiting the device; the distal portion may define a concave surface; the plurality of substantially flat crystals may be arranged on the concave surface; the distal portion may define a cavity for containing a fluid; energy emitted by the plurality of flat crystals may travel through the sonolucent cover prior to exiting the device; the common focal point may be a common focal area; adjusting a distance of the common focal point relative to the distal portion; adjusting a position of the common focal point relative to the distal portion; and the adjusting may include adjusting at least some of the plurality of flat crystals relative to other portions of the distal portion.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a partial schematic view of a device according to an embodiment of the invention.

FIG. 2 is a schematic view of a device according to another embodiment of the invention.

FIG. 3 is a partial schematic view of an elongate portion of a device according to a further embodiment of the invention.

FIG. 4 is a schematic view of a distal portion of a device according to yet another embodiment of the invention.

FIG. 5 is a schematic view of a distal portion of a device according to a yet further embodiment of the invention.

FIG. 6 is a schematic view of the device of FIG. 1 including the distal portion of FIG. 5 disposed in a body lumen according to still another embodiment of the invention.

FIG. 7 is a schematic view of the device of FIG. 1 including the distal portion of FIG. 4 disposed in a body lumen according to a still further embodiment of the invention.

FIGS. 8A and 8B are schematic views of a distal portion of a device according to another embodiment of the invention.

FIG. 9 is a schematic view of a distal portion of a device according to a further embodiment of the invention.

FIG. 10A is a schematic view of a device according to yet another embodiment of the invention.

FIG. 10B is a cross-sectional view of the device of FIG. 1OA along line X-X of FIG. 10A.

### DESCRIPTION OF THE EMBODIMENTS

Reference will now be made in detail to the exemplary embodiments of the invention, examples of which are illustrated in the accompanying drawings. Wherever possible, the same reference numbers will be used throughout the drawings to refer to the same or like parts.

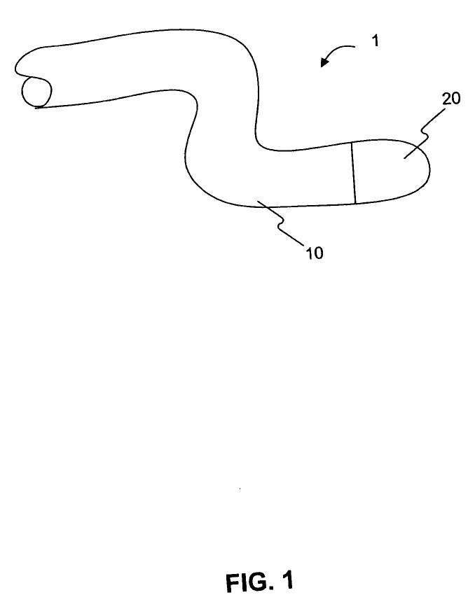

FIG. 1 depicts an example of a medical device 1 according to the present invention. Medical device 1 includes elongate portion 10 and distal portion 20.

Each of medical device 1, elongate portion 10, and distal portion 20 may have any suitable dimensions. For example, one or more of medical device 1, elongate portion 10, and distal portion 20 may have a diameter less than or equal to about 10 millimeters (e.g., less than or equal to about 8 millimeters) such that medical device 1, elongate portion 10, and distal portion 20 may be placed in a naturally existing body orifice in a minimally invasive manner.

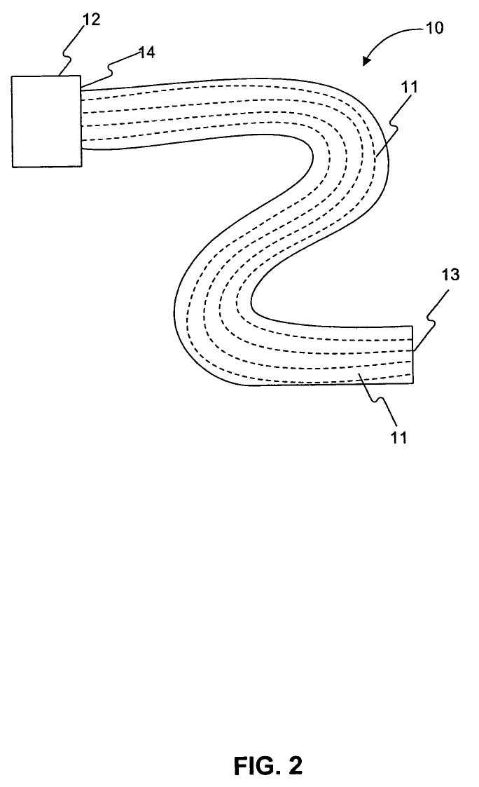

An example of an elongate portion 10 is depicted in FIG. 2. Elongate portion 10 may be any elongate portion configured to be used in a medical procedure. For example, elongate portion 10 may have any length, width, diameter, and cross-sectional shape, and may be made of any suitable material to be placed in and/or around a body portion (e.g., body cavity and/or body tissue). Elongate portion 10 may be flexible, for example, so as to follow the tortuous anatomy of a body lumen, or may be substantially rigid, for example, so as to be more easily advanced into a body cavity. Elongate portion 10 may be attached and/or operatively connected at its proximal end 14 to a handle or controller 12. Controller 12 may be any suitable handle or actuator known in the art and may be configured to control elongate portion 10 and/or distal portion 20. A distal end 13 of elongate portion 10 may be configured to accommodate, be operatively connected to, or integral with distal portion 20.

Elongate portion 10 may include one or more lumens 11 extending therethrough. Lumens 11 may extend through any portion of elongate portion 10. For example, at least one lumen 11 may be substantially coaxial with elongate portion 10. In another example, at least one lumen 11 may extend through only a portion of elongate portion 10. In a further example, at least a portion of at least one lumen 11 may be disposed in and substantially parallel to a longitudinal axis of elongate portion 10. In yet another example, at least one lumen 11 may emerge from any portion of elongate portion 10 (e.g., distal end 13 or a side of elongate portion 10). At least one lumen 11 may emerge from a side of elongate portion proximal to distal end 13.

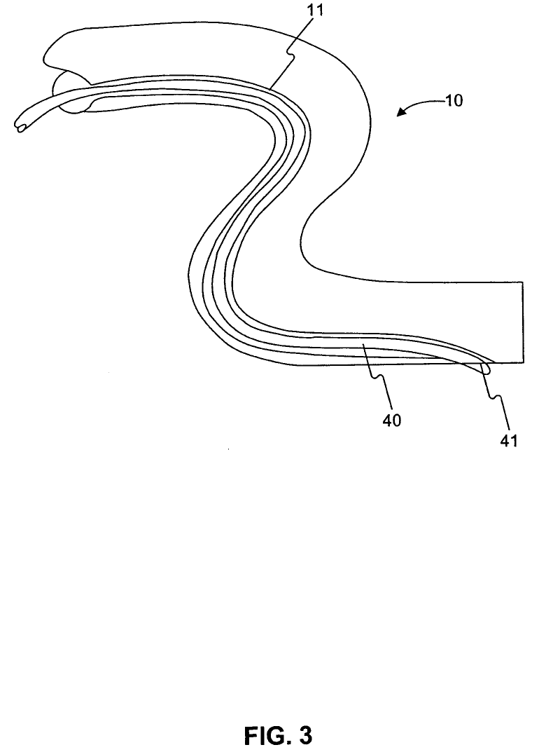

At least one lumen 11 may have any suitable configuration and/or cross-section. For example, as shown in FIG. 3, at least one lumen 11 may be configured to accommodate a medical device therethrough (e.g., a fiber optic device 40 including a visualization portion at its distal end 41, graspers, cutters, needles, knives, and/or forceps). Fiber optic device 40 may have any suitable dimensions, for example, a diameter less than or equal to 3 millimeters. Distal end 41 of fiber optic device 40 may be disposed so as to allow direct visual access to the body cavity and/or body lumen within which device 1 is disposed. For example, most of fiber optic device 40 may be disposed in a lumen 11 in a manner substantially parallel to the longitudinal axis of elongate portion 10. Distal end 41 may then be directed away from the longitudinal axis of elongate portion 10 such that distal end 41 emerges from a side of elongate portion 10. Distal end 41, when emerging from the side of elongate portion 10, may be directed to a specific location relative to any portion of device 1, for example, a location adjacent to distal portion 20 (e.g., point 23 as described herein). Visual access to the body cavity and/or body lumen via distal end 41 of fiber optic device 40 may allow more effective use of device 1 and its distal portion 20 in a medical procedure, for example, a transcervical treatment of submucosal uterine fibroids. Distal end 41 of fiber optic device 40 may include a lens angle and/or be configured to increase a total field of view of fiber optic device 40 if distal end 41 and/or another portion of fiber optic device 40 (e.g., a handle) is rotated. Such a configuration may minimize the amount of bending and stresses that fiber optic device 40 may be subjected to.

Fiber optic device 40 may be disposable along with the rest of device 1, for example, if fiber optic device 40 is manufactured in a relatively cost-effective manner. However, fiber optic device 40 may also be reusable, for example, by removing fiber optic device 40 from lumen 11 after completing a medical procedure including device 1. Fiber optic device 40 may then be sterilized and used in another medical procedure, for example, in a lumen 11 of another medical device 1.

In another example, at least one lumen 11 may have a varying cross-sectional shape and/or cross-sectional area. In a further example, at least one lumen 11 may be configured to accommodate wires and/or other electronic components therethrough. Such wires or others components may extend between controller 12 and distal portion 20.

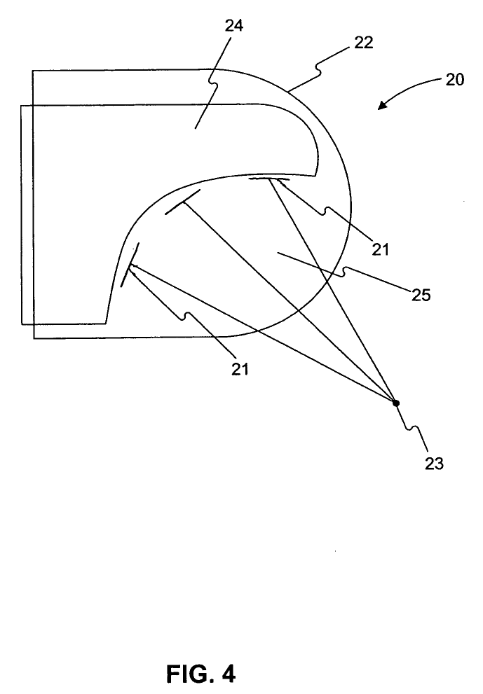

As shown most clearly in the various embodiments of FIGS. 4, 5, and 9, distal portion 20 may include a housing 24, one or more discrete flat crystals 21, and a covering 22 disposed in any suitable configuration. Flat crystals 21 may be substantially flat, and may have a small thickness in comparison to its length and width. For example, flat crystals 21 may be substantially flat in one dimension, and may have any suitable shape (e.g., circle, square, etc.) in the other dimension(s) (e.g., length and/or width). Flat crystals 21 may be made of any suitable energy emitting material, for example, one or more piezoelectric ceramics. One example of such a piezoelectric ceramic is Lead Zirconate Titanate (PZT). There are several kinds of PZTs available, for example, PZT-2, PZT-4, PZT-5, PZT-6, PZT-7, and PZT-8. PZT-8 may be used in this or other applications where a high acoustic power may be generated.

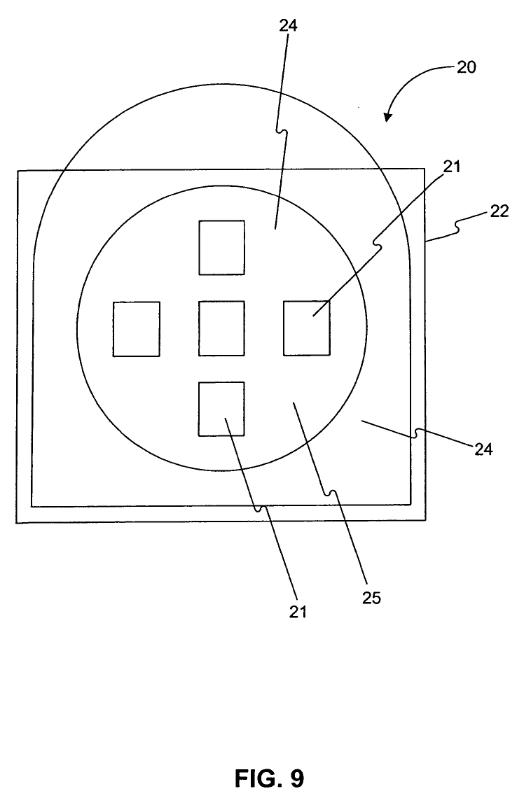

Flat crystals 21 may be configured and/or arranged in housing 24 such that energy emissions from the flat crystals 21 may be focused on a focal point external to distal portion 20. For example, flat crystals 21 may be configured and/or arranged relative to cavity 25 such that ultrasound emissions from the flat crystals 21 may be focused on point 23 which is located external to distal portion 20 Flat crystals 21 may be arranged in a two-dimensional array or a three-dimensional array, such as a 3-D array shown in FIG. 9. For example, when distal portion 20 is in contact with and/or adjacent to tissue, point 23 may be disposed below a surface of the tissue. The ultrasound energy focused on point 23 may assist in treating the tissue, for example, by providing energy to the tissue.

Housing 24 may be a discrete portion connected to elongate portion 10 or may be an extension of and integral with elongate portion 10. A portion of housing 24 may be concave and at least partially define cavity 25. The cavity 25 also may be at least partially defined by a covering 22, to be described further herein. The concave surface of housing 24 may have any suitable configuration, for example, the concave surface may be substantially smooth and/or may have a plurality of flat surfaces disposed and/or joined at angles relative to each other (e.g., like a soccer ball or buckyball). The concave surface of housing 24 may be configured to accommodate flat crystals 21 therein and also may accommodate a fluid within cavity 25. For example, if concave surface of housing 24 includes a plurality of flat surfaces disposed and/or joined at relative angles to each other, at least some of the plurality of flat surfaces may be configured to accommodate one or more flat crystals 21. One or more lumens 11 may be in flow communication with cavity 25 and may be configured to deliver and/or remove fluid from cavity 25, for example, during use of device 1 in a medical procedure. Cavity 25 may also or alternatively have fluid placed within it during manufacturing and/or may not be connected to one or more lumens 11, instead having a fixed volume of fluid disposed within it during sale, storage, and use.

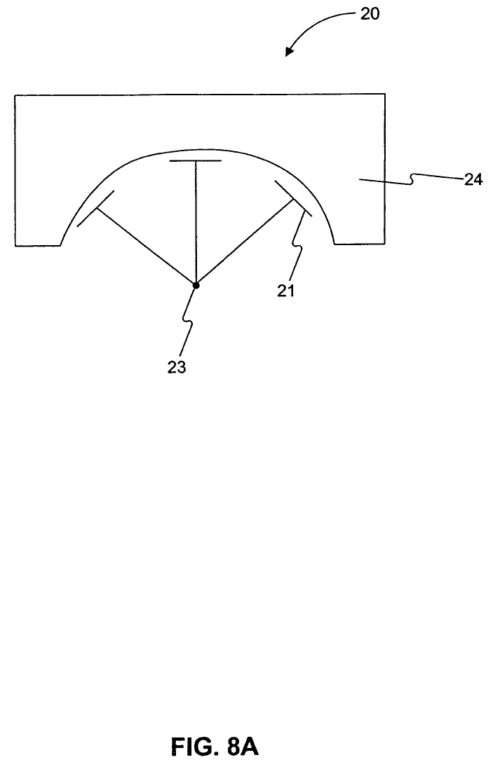

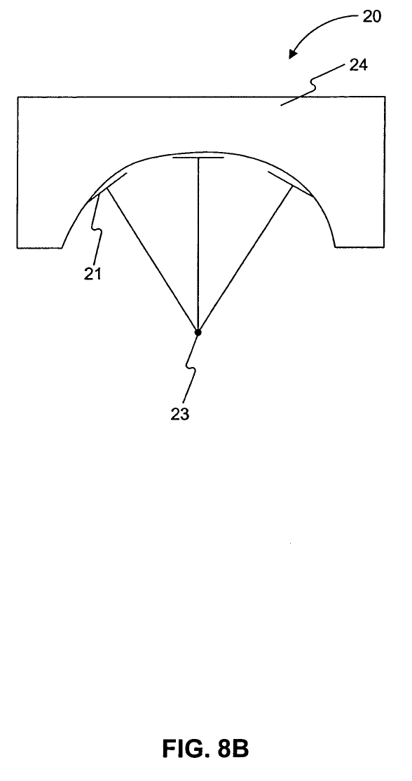

Flat crystals 21 may be affixed to a surface of housing portion 24. Flat crystals 21 may be positioned relative to each other to adjust point 23 relative to one or more of housing 24, covering 22, and the tissue to be treated. Flat crystals 21 may be operatively connected to controller 12 and/or a source of ultrasound energy located at a proximal end of device 1. Controller 12 and/or the source of ultrasound energy may be connected to flat crystals 21 via wires and/or other electronic components disposed within lumen 11 or other suitable components such as wireless transmitters. Each of flat crystals 21 may also be configured to be independently adjusted relative to other flat crystals 21 so as to dynamically adjust a position of point 23. In such a configuration, flat crystals 21 may be connected to housing 24, but may not have a fixed position relative to housing 24. For example, flat crystals 21 may be rotated about an axis such that the angular position between flat crystals 21 and one or more of housing 24, covering 22, and the tissue to be treated is changed. An example of such a progression is shown in FIGS. 8A and 8B. In FIG. 8B, the outer flat crystals 21 b have been rotated away from the inner flat crystal 21 a such that point 23 has moved away from distal portion 20, at least as compared to the configuration in FIG. 8A. Flat crystals 21 a, 21 b, however, still maintain a common focal point. In various embodiments, any of flat crystals 21 may be moved in any direction, together or independently, relative to each other so as to move point 23 in any direction relative to distal portion 20.

Using flat crystals 21, as opposed to more complex-shaped crystals, in device 1 is preferable because flat crystals 21 may be easier and less expensive to manufacture than more complex-shaped crystals. This lowers the cost of producing device 1 so that device 1 may be disposable. The disposability of device 1 is desirable, for example, because once device 1 has been used in a medical procedure, the sterility of device 1 may be compromised. Thus, it is preferable to dispose of device 1 rather than risk using contaminated device 1 in another procedure.

Covering 22 may be disposed around housing 24 and flat crystals 21 and may be configured to seal flat crystals 21 in a cavity 25 of distal portion 20 in a substantially fluidtight manner. For example, covering 22 may be a sonolucent covering, shaped like a dome, for example, that is connected to housing 24 in a substantially fluidtight manner. A sonolucent covering includes, for example, any suitable material that allows passage of ultrasonic waves therethrough without substantial energy loss due to reflection or attenuation. Reflection may be minimized, for example, if the acoustic impedance of the material is close to the acoustic impedance of water (e.g., about 1.5 Megarayls). Both reflection and attenuation may be minimized, for example, if the thickness of covering 22 is much smaller (e.g., less than or equal to 1/10th) than the wavelength of covering 22. For example, if the sound velocity (V) in covering 22 is about 2,500 meters per second and the operating frequency (F) of flat crystals 21 is about 2.5 MHz, then the wavelength of covering 22 may be derived by the formula λ=V/F=1.0 mm. Accordingly, using those numbers, a covering 22 having a thickness of equal to or less than about 1.0 mm (e.g., less than or equal to about 0.1 mm or about 0.004 inches) may be sonolucent. Thus, in general, the higher the operating frequency of the flat crystals 21, the thinner covering 22 may need to be. Covering 22 may be configured to allow passage of ultrasonic waves therethrough whether the ultrasonic waves are being passed through a fluid (e.g., water) or tissue. For example, covering 22 may be thin enough such that covering 22 does not interfere with ultrasound waves passing therethrough. Covering 22 may have a thickness less than or equal to about 0.001 inches. Covering 22 is preferably clear, but may be opaque and/or have opaque portions.

Covering 22 may be made of any suitable material, for example, a thin-film polymer such as polyethylene terephthalate (PET), polyethylene (PE), polypropylene (PP), and/or nylon. PE and/or nylon may have an acoustic impedance between about 1.7 Megarayls and 3.0 Megarayls. Covering 22 may be made of a rigid material configured to be pressed into the tissue to maximize contact and coupling, or covering 22 may be made of a more compliant material configured to conform to body anatomy. A more compliant material for covering 22 may also allow a focal depth to be changed by changing pressure on crystals 21. For example, covering 22 may allow the location of point 23 to be changed by changing fluid pressure in cavity 25.

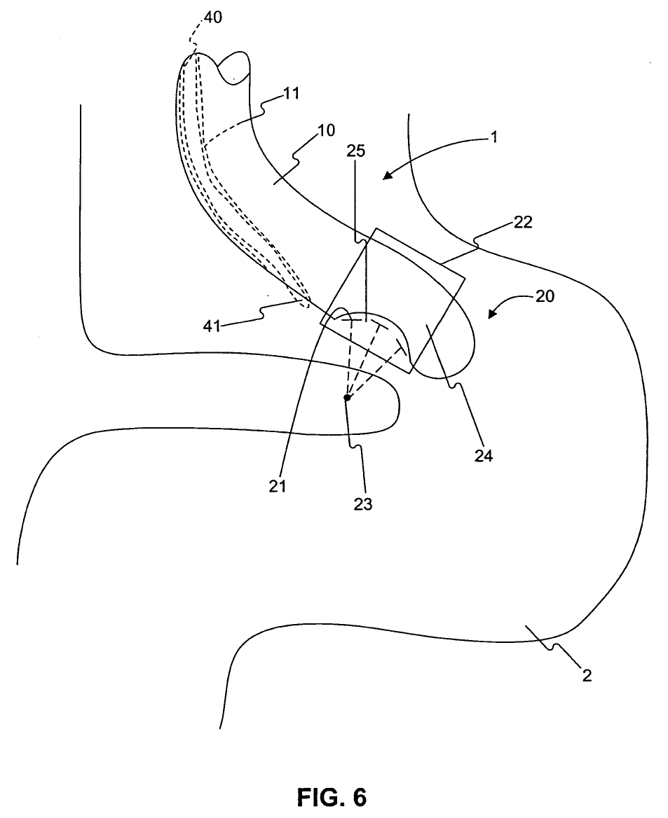

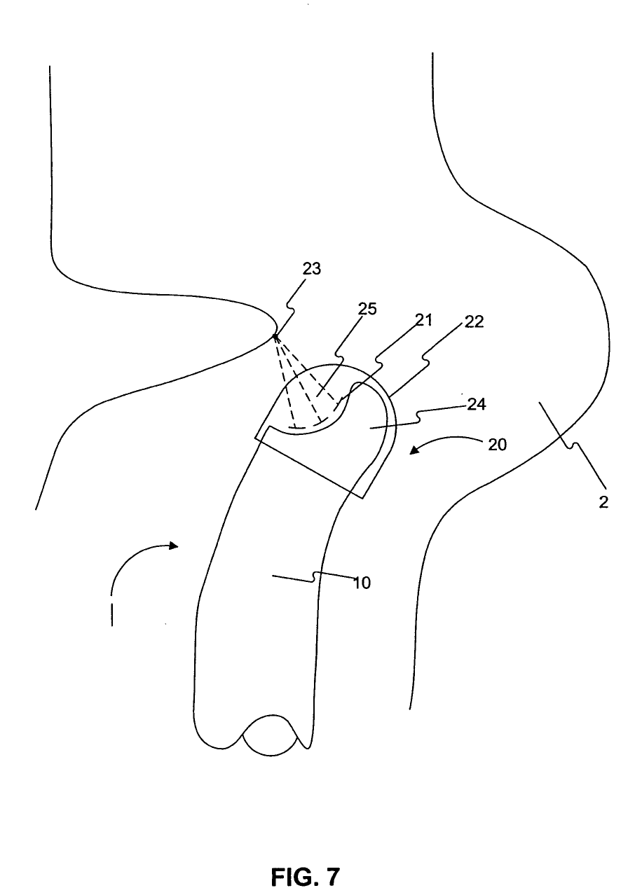

Covering 22 may have any suitable configuration. For example, covering 22 may have a sock-like configuration as shown in FIG. 7. In such a configuration, covering 22 may have one open distal end, and housing 24 may be placed within covering 22 via the open distal end such that covering 22 is covering cavity 25. Covering 22 generally defining the open distal end may then be attached to housing 24 using any suitable method. In general, covering 22 may be attached to housing 24 using any suitable method, for example, via an adhesive, an ultrasonic weld, and/or a mechanical clamp. In another example, covering 22 may have a tube-like configuration as shown in FIG. 6. In such a configuration, covering 22 may have two open ends, and a distal end of housing 24 may be placed through one of the open ends and out the other open end such that covering 22 is covering cavity 25. Covering 22 generally defining the two open ends may then be attached to housing 24 using any suitable method. In a further configuration, covering 22 may have a shape (e.g., a circle and/or ovaloid shape) and/or surface area sufficient to cover cavity 25, and then the outer edge(s) of covering 22 may be attached to housing 24 using any suitable method. In such configurations, fluid may not accidentally enter the patient by exiting cavity 25.

Covering 22 may be made using any suitable method. For example, covering 22 may be blow-molded into the sock-like configuration shown in FIG. 7. In another example, covering 22 may be extruded as a thin walled elongate tubular member, and then may be cut so as to form the tube-like configuration shown in FIG. 6. In a further example, covering 22 may be extruded as a thin flat film, and then a section may be cut from the thin flat film so as to form covering 22. Ends of covering 22 may then be attached to each other using any suitable method, resulting in the tube-like configuration shown in FIG. 6. Alternatively, covering 22 may be rolled around the appropriate section of housing 24 (e.g., so as to cover cavity 25), and then ends of this covering 22 may be attached to each other and/or attached to housing 24 using any suitable method, possibly resulting in the tube-like configuration shown in FIG. 6.

Fluid may be disposed in cavity 25, for example, to provide energy coupling to the tissue and may assist in cooling the tissue when device 1 is in contact with and/or disposed adjacent to tissue. The fluid may transmit the energy with minimal or no deflection so that substantially the same amount of energy emitted from the cavity is delivered to common focal point and/or the body tissue to be treated. Such deflection may be minimized, for example, because at the face of flat crystal 21, the fluid may create a perfect mechanical contact with the surface of the flat crystal 21. For the tissue, the acoustic properties of the fluid (e.g., impedance) may match up very well with the acoustic properties of tissue. Accordingly, there may not be any energy reflection at the fluid-tissue interface (e.g., covering 22). In various embodiments, any reflection due to an impedance mismatch between flat crystals 21 and the fluid may be significantly reduced by applying matching layers on the surface of flat crystals 21.

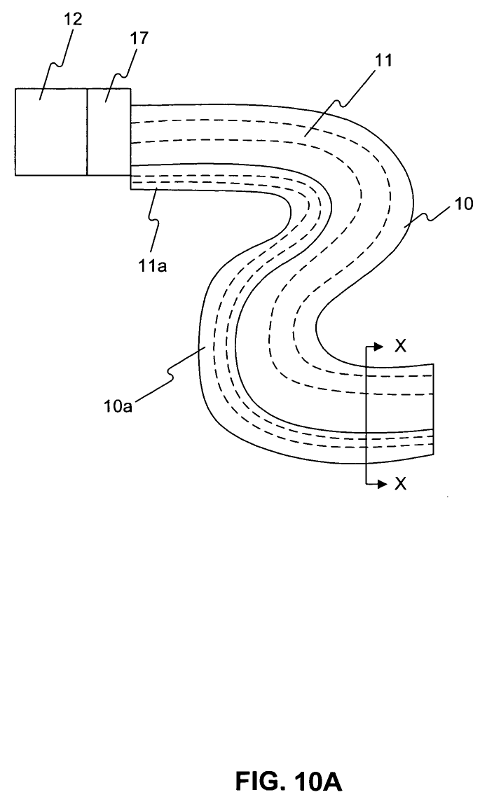

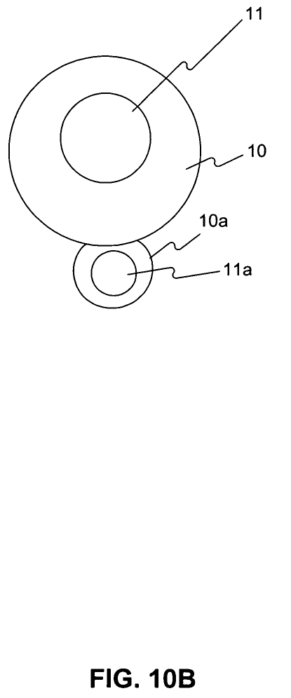

Covering 22 may be disposed completely around housing 24 or may be disposed around only a portion of housing 24. For example, covering 22 may be attached in a substantially fluidtight manner to the portion of the outer surface of housing 24 that defines cavity 25. One or more lumens 11 may be in flow communication with cavity 25 and may be configured to deliver and remove fluid from cavity 25, for example, to provide a circulation of fluid during a procedure. The distal end of one or more lumens 11 may be connected to cavity 25 while a proximal end of lumens 11 may be connected to a controller 12 and/or a source of fluid. Any suitable fluid may be used, for example, water and/or a saline solution. A temperature of the fluid may be regulated (e.g., held substantially constant), for example, to provide a repeatable and consistent result. The temperature of the fluid may be regulated using any suitable method, for example, by heating and/or cooling the fluid via a temperature regulator 17 associated with one or more lumens 11, controller 12, and/or the source of fluid as shown in FIG. 10A.

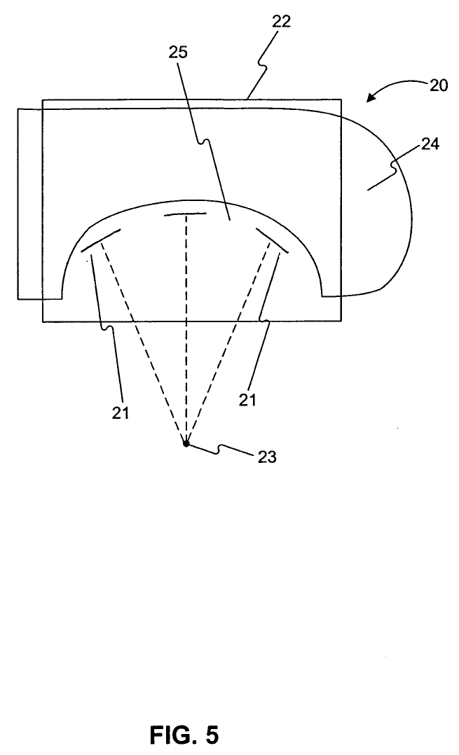

Distal portion 20 may have any suitable configuration. For example, as shown in FIG. 5, distal portion 20 may be configured to emit energy to a side of elongate portion 10, distal portion 20, and/or any other portion of device 1. In other words, a center line of the energy being emitted from distal portion 20 may be substantially perpendicular to a longitudinal axis of one or more of device 1, elongate portion 10, and/or distal portion 20. Covering 22 may be substantially in the shape of a cylinder which extends sufficiently proximally and distally along distal portion 20 and/or housing 24 such that its proximal and distal ends extend past the proximal and distal extremities of cavity 25, respectively. Accordingly, a combination of housing 24 and covering 22 cooperate to define cavity 25 and also to confine a fluid to cavity 25. Flat crystals 21 are disposed in cavity 25, for example, they may be disposed along and/or attached to a surface of housing 24 defining cavity 25. Device 1 having a configuration as shown in FIG. 5 may be desirable, for example, in the transurethral treatment of prostate conditions such as prostate cancer or benign prostatic hyperplasia.

In the exemplary configuration of FIG. 4, distal portion 20 may be configured to emit energy at any suitable angle, for example, an angle that is not perpendicular to a longitudinal axis of elongate portion 10, distal portion 20, and/or any other portion of device 1. In such a configuration, point 23 may be located distal to the distalmost point of device 1. Such an angle may be desired, for example, in a situation where the tortuous anatomy and/or shape of a body cavity prevents device 1 from focusing energy to a point 23 within the desired tissue to be treated, were the emission of energy limited to a side of device 1. Covering 22 may be substantially in the shape of a contiguous cylinder and/or dome which extends sufficiently proximally along distal portion 20 and/or housing 24 such that its proximal ends extend past the proximal extremity of cavity 25. Accordingly, a combination of housing 24 and covering 22 cooperate to define cavity 25 and also to confine a fluid to cavity 25. Flat crystals 21 are disposed in cavity 25, for example, they may be disposed along and/or attached to a surface of housing 24 defining cavity 25.

FIG. 9 shows a distal portion 20 having a plurality of flat crystals 21 arranged in a cross-like configuration. However, distal portion 20 may have any number of flat crystals 21 and may be arranged in any desired configuration. Flat crystals 21 have a common focal point, for example, point 23.

Device 1 may be used in any suitable medical or non-medical procedure. For example, device 1 may be used in transcervical treatment of uterine fibroids, transrectal treatment of prostate conditions, transurethreal treatment of prostate conditions, laparascopic treatment of endometriosis lesions, non-incisional or incisional treatments of pelvic floor tissue, transurethral treatment of pelvic floor tissue for stress urinary incontinence, and/or cosmetic treatment of wrinkles.

Embodiments of the invention include a method of using device 1. In such a method, device 1 may be provided. Device 1 may have any suitable distal portion 20 given the medical procedure to be performed. For example, distal portion 20 may have a configuration as shown in FIGS. 4, 5, or 9. Device 1 may or may not have a lumen 11 configured to accommodate a medical device, for example, fiber optic device 40. Fiber optic device 40 may be integral with device 1, or may be provided separately from device 1 and inserted within lumen 11. In such a case, fiber optic device 40 may be placed through lumen 11 until it emerges from the distal end of lumen 11 and/or a side of elongate portion 10. Distal end 41 of fiber optic device 40 may be aimed at a location adjacent to distal portion 20, for example, approximately at point 23. In various embodiments, fiber optic device 40 may be disposed on lumen 11a, which is defined by an elongate member 10a which is external and/or attached to elongate member 10, for example, as shown in FIGS. 10A-10B. Elongate member 10a may be substantially parallel to and/or may have substantially the same length as elongate member 10.

Device 1 may be advanced into a body lumen 2, for example, as shown in FIGS. 6 and 7. Fiber optic device 40 may be advanced into lumen 11 of elongate portion 10 via a fitting disposed at a proximal end of lumen 11. The fitting may be a passive gasket and/or O-ring that may or may not be fluid sealable, and may be configured to prevent biological material from leaking back through the fitting. Advancing fiber optic device 40 through lumen 11 may guide fiber optic device 40 to a distal exit port at or near the end of lumen 11, out of lumen 11 and/or elongate portion 10, and into body lumen 2.

If device 1 includes fiber optic device 40, distal end 41 may be used to assist in advancing device 1 into the body lumen 2 and positioning device 1 so that device 1 may be used to treat the desired body portion. Positioning device 1 may include positioning and/or rotating distal portion 20 such that distal portion 20 is properly arranged relative to the desired body portion. Once positioned, flat crystals 21 may be activated via controller 12 such that energy (e.g., ultrasound waves) are emitted from flat crystals 21, travel through the fluid disposed in cavity 25 without being substantially disrupted, travel through covering 22 without being substantially disrupted, and become focused on point 23. Point 23 may be located at the body tissue to be treated. Point 23 may be located on the surface of the body tissue, for example, as shown in FIG. 7, or may be located below the surface of the body tissue, for example, as shown in FIG. 6. The energy (e.g., ultrasound waves) focused on point 23 may be used to treat the tissue at point 23 for any medical purpose, such as removal, incision, necrosis, thermal treatment, or otherwise destroying tissue. Device 1 and/or point 23 may be moved and/or repositioned as necessary to achieve the desired medical effect. If device 1 has a configuration as shown in FIGS. 8A and 8B, point 23 may be suitably adjusted relative to any of device 1, elongate portion 10, and distal portion 20. Once the treatment has been completed, device 1 may be removed and disposed of. If fiber optic device 40 is to be reused, fiber optic device 40 may be removed from lumen 11 prior to disposal of device 1.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device, comprising:
an elongate portion; and
a distal portion including a plurality of substantially flat crystals configured to emit energy to a common focal point.

2. The medical device of claim 1, wherein the plurality of substantially flat crystals are arranged on a concave surface of the distal portion.

3. The medical device of claim 1, wherein the distal portion includes a sonolucent cover.

4. The medical device of claim 3, wherein the sonolucent cover covers the plurality of substantially flat crystals.

5. The medical device of claim 1, wherein the common focal point is located adjacent to a side of the distal portion.

6. The medical device of claim 1, wherein the common focal point is located distal to the distal portion.

7. The medical device of claim 1, wherein the distal portion defines a cavity for containing a fluid.

8. The medical device of claim 7, wherein the elongate portion includes one or more lumens configured to circulate fluid to the cavity.

9. The medical device of claim 7, wherein the sonolucent cover defines at least a portion of the cavity.

10. The medical device of claim 7, wherein a concave surface of the distal portion defines at least a portion of the cavity, the plurality of substantially flat crystals being arranged on the concave surface.

11. The medical device of claim 1, wherein the elongate portion includes a lumen configured to accommodate a fiber optic device.

12. The medical device of claim 11, wherein a distal end of the lumen is disposed on a side of the elongate portion.

13. The medical device of claim 11, further comprising a fiber optic device disposed in the lumen.

14. The medical device of claim 13, wherein an area around the distal portion is visible via the fiber optic device.

15. The medical device of claim 1, wherein the energy is ultrasound energy.

16. The medical device of claim 1, wherein the elongate portion is flexible so as to traverse tortuous anatomy of a patient.

17. The medical device of claim 1, wherein the plurality of substantially flat crystals are arranged in a two-dimensional array.

18. The medical device of claim 1, wherein the plurality of substantially flat crystals are arranged in a three-dimensional array.

19. A medical device, comprising:
a distal portion including a plurality of discrete substantially flat crystals configured to emit ultrasound energy to a common focal point to treat the tissue;
a proximal portion configured to control the emission of ultrasound energy; and
an elongate flexible potion connecting the proximal portion to the distal portion.

20. The medical device of claim 19, wherein the plurality of substantially flat crystals are arranged on a concave surface of the distal portion.

21. The medical device of claim 19, wherein the distal portion includes a sonolucent cover.

22. The medical device of claim 21, wherein the sonolucent cover covers the plurality of substantially flat crystals.

23. The medical device of claim 19, wherein the common focal point is located adjacent to a side of the distal portion.

24. The medical device of claim 19, wherein the common focal point is located distal to the distal portion.

25. The medical device of claim 19, wherein the distal portion defines a cavity for containing a fluid.

26. The medical device of claim 25, wherein the elongate portion includes one or more lumens configured to circulate fluid to the cavity.

27. The medical device of claim 25, wherein the sonolucent cover defines at least a portion of the cavity.

28. The medical device of claim 25, wherein a concave surface of the distal portion defines at least a portion of the cavity, the plurality of substantially flat crystals being arranged on the concave surface.

29. The medical device of claim 19, wherein the elongate portion includes a lumen configured to accommodate a fiber optic device.

30. The medical device of claim 29, wherein a distal end of the lumen is disposed on a side of the elongate portion.

31. The medical device of claim 29, further comprising a fiber optic device disposed in the lumen.

32. The medical device of claim 31, wherein an area around the distal portion is visible via the fiber optic device.

33. The medical device of claim 19, wherein the energy is ultrasound energy.

34. The medical device of claim 19, wherein the elongate portion is flexible so as to traverse tortuous anatomy of a patient.

35. The medical device of claim 19, wherein the plurality of substantially flat crystals are arranged in a two-dimensional array.

36. The medical device of claim 19, wherein the plurality of substantially flat crystals are arranged in a three-dimensional array.

37. A method of treating tissue, comprising:
providing a medical device including an elongate portion and a distal portion, the distal portion including a plurality of substantially flat crystals configured to emit energy to a common focal point;
advancing the medical device into a body lumen;
providing energy to a body tissue via the substantially flat crystals; and
removing the medical device from the body lumen.

38. The method of claim 37, wherein the medical device includes a fiber optic device, and further comprising visualizing an area around the distal portion via the fiber optic device.

39. The method of claim 37, further comprising:
providing a fiber optic device;
advancing the fiber optic device through a lumen in the elongate portion; and
visualizing an area around the distal portion via the fiber optic device.

40. The method of claim 39, further comprising determining an area of body tissue to be treated; and
positioning the medical device in the body lumen so as to deliver energy to the area of body tissue to be treated,
wherein the positioning occurs during the visualizing.

41. The method of claim 37, wherein the energy is provided to an area on a surface of the body tissue.

42. The method of claim 37, wherein the energy is provided to an area below a surface of the body tissue.

43. The method of claim 37, wherein the energy is provided to an area distal to the distal portion.

44. The method of claim 37, wherein the energy is provided to an area proximal to a distalmost point of the distal portion.

45. The method of claim 37, further comprising adjusting a position of the plurality of flat crystals relative to other portions of the distal portion while the medical device is disposed in the body lumen.

46. The method of claim 37, wherein the energy is ultrasound energy.

47. The method of claim 37, wherein the plurality of substantially flat crystals are disposed in a cavity of the medical device.

48. The method of claim 47, further comprising providing fluid to the cavity.

49. The method of claim 37, wherein the distal portion includes a sonolucent cover.

50. The method of claim 49, wherein the sonolucent cover covers the plurality of substantially flat crystals.

51. The method of claim 50, wherein energy emitted by the plurality of flat crystals travels through the sonolucent cover prior to exiting the device.

52. The method of claim 37, wherein the distal portion defines a concave surface.

53. The method of claim 52, wherein the plurality of substantially flat crystals are arranged on the concave surface.

54. The method of claim 37, wherein the distal portion defines a cavity for containing a fluid.

55. The method of claim 54, wherein energy emitted by the plurality of flat crystals travels through the sonolucent cover prior to exiting the device.

56. The medical device of claim 7, further comprising a temperature regulator configured to regulate a temperature of the fluid.

57. The medical device of claim 1, wherein the elongate portion is flexible so as to provide sufficient angulation with respect to the wall of a body cavity.

58. The medical device of claim 1, further comprising another elongate portion external to the elongate portion,
wherein the another elongate portion includes a lumen.

59. The medical device of claim 1, wherein the elongate portion includes a lumen configured to accommodate at least one a grasper, a cutter, a needle, a knife, and forceps.

60. The medical device of claim 25, further comprising a temperature regulator configured to regulate a temperature of the fluid.

61. The medical device of claim 19, wherein the elongate portion is flexible so as to provide sufficient angulation with respect to the wall of a body cavity.

62. The medical device of claim 19, further comprising another elongate portion external to the elongate portion,
wherein the another elongate portion includes a lumen.

63. The medical device of claim 19, wherein the elongate portion includes a lumen configured to accommodate at least one a grasper, a cutter, a needle, a knife, and forceps.
